(19) United States
(12) Patent Application Publication
(10) Pub. No.: US 2007/0178544 A1
(43) Pub. Date: Aug. 2, 2007

(54) Anti fk778antibodies and high sensitive immunoassay methods

(76) Inventors: Kouichi Tamura, Tokyo (JP); Takeshi Kato, Tokyo (JP); Kenji Tabata, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/591,287

(22) PCT Filed: Feb. 28, 2005
(86) PCT No.: PCT/JP05/03819

§ 371 Date: Feb. 2, 2007

(30) Foreign Application Priority Data
Mar. 5, 2004 (AU) 20049011191

(51) Int. Cl. G01N 33/53; C07K 16/18; C12N 5/06

(52) U.S. Cl. 435/7.92; 435/326; 530/388.1

(57) ABSTRACT

This invention relates to antibodies capable of binding to the FK778 substance, to a highly-sensitive immunoassay method, which utilizes an antibody for the FK778 substance, and to a test kit for measuring the concentration of the FK778 substance.

### TECHNICAL FIELD

This invention relates to novel antibodies, a highly-sensitive immunoassay method and a test kit for practicing this method. More particularly, this invention relates to antibodies capable of binding to the FK778 substance, to a highly-sensitive immunoassay method, which utilizes an antibody for the FK778 substance, and to a test kit for measuring the concentration of the FK778 substance.

### Background Art

The FK778 substance is derived from an active leflunomide-metabolite, A77 1726 and has high immunosuppressive effect. It is known that the said compound has the following structural formula (PCT/JP03/04722): FK778

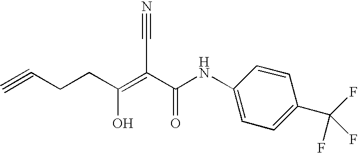

The FK778 substance, in very small doses, shows very potent immunosuppressive activity. Therefore, for effectively and continuously suppressing the rejection reaction on the occasion of transplantation, for example organ transplantation, a simple and easy technique is required which will enable highly-sensitive and bedside monitoring of the blood concentration of the said compound after administration thereof to living bodies. For such monitoring, to establish a technique for precise and practical determination of very low concentration of the said compound is thought to be of very great importance.

The so-far used methods of assaying the small amounts of low-molecular weight substances contained in biological samples and the like include gas chromatography, high-performance liquid chromatography, radioimmunoassay and enzyme immunoassay and so on.

However, these methods are disadvantageous in some sense or other, for example, (1) the procedure is complicated, and (2) a large-sized apparatus is required.

The purpose of the present invention is to develop substance and system for measuring the FK778 substance in a simple and easy manner.

As a result of intensive investigations to solve the above problems, the present inventors succeeded in obtaining an antibody capable of binding to the FK778 substance. Then, the inventors investigated efficacy of the antibody in immunological assay method, and found that the antibody is very useful as a reagent for measurement of the FK778 substance.

### BRIEF DESCRIPTION OF THE DRAWING

FIG. 1 shows concentration-response curve for FK778 and FR271764 inhibitory effect on binding of monoclonal antibody 7A to FR271764-BSA.

FIG. 2 shows concentration-response curve for FK778 and FR271764 inhibitory effect on binding of monoclonal antibody 9A to FR271764-BSA.

FIG. 3 shows concentration-response curve for FK778 and FR271764 inhibitory effect on binding of monoclonal antibody 20A to FR271764-BSA.

### SUMMARY OF THE INVENTION

In the present invention, antibodies capable of binding to the FK778 substance, a highly-sensitive immunoassay method, which utilizes an antibody for the FK778 substance, and a test kit for measuring the concentration of the FK778 substance, are provided.

In the following, the present invention is described in further detail.

(I) An Antibody Capable of Binding to the FK778 Substance

The above-mentioned antibody includes a polyclonal antibody and a monoclonal antibody.

The immunogens for obtaining polyclonal or monoclonal antibodies include the above FK778 and derivative thereof as follows:
FR270531 (FK778-oxyhexanoic acid pentafluorophenyl ester): 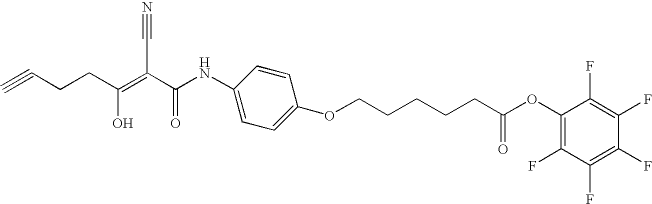
FR267471 (FK778-oxyhexanoic acid): 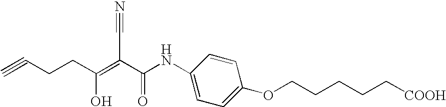
FR266831 (FK778-glutaric acid): 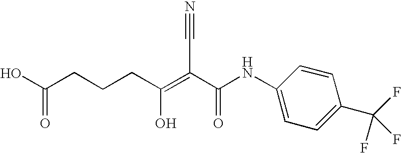
FR271764 (M-III):

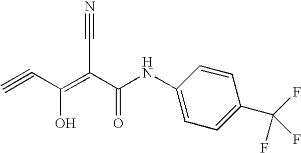

The polyclonal antibody may be classified according to its H chain (heavy chain) into such classes as IgG, IgA, IgM, IgD or IgE and further into subclasses of each class. They may be of any class if they can bind to the FK778 substance. A particularly preferred class is IgG.

The polyclonal antibody is purified from its antiserum obtained by immunizing an animal with an immunogen such as above.

The immunization step is carried out by a conventional method.

There is no particular limitation as to the animal species to be immunized. Generally, rabbits, guinea pigs, rats, mice, goats and the like are used. The substance to serve as immunogen is generally used in the form of a conjugate with a carrier such as bovine serum albumin (hereinafter referred to as BSA), bovine thyroglobulin, gelatin or hemocyanine so that the immunogenicity can be increased. Such conjugate with BSA (BSA-immunogen conjugate) can be obtained, for example, by converting the immunogen substance to a half ester of a dicarboxylic acid such as succinic acid, then reacting the half ester with N-hydroxysuccinimide or the like in the presence of a condensing agent such as dicyclohexylcarbodiimide and further reacting the resulting activated ester with BSA.

The polyclonal antibody is purified from the thus-obtained antiserum by conventional means such as salting out with ammonium sulfate or the like, centrifugation, dialysis and column chromatography.

Although the monoclonal antibody may be classified according to its H chain as in the case of polyclonal antibody, any type of monoclonal antibodies can be utilized, as long as it can bind to the FK778 substance. A particularly preferred class is IgG.

The monoclonal antibody is generally produced by the technique of cell fusion and cloning. It can also be produced by using genetic engineering techniques.

The antibody-producing cells to be used in the step of cell fusion (e.g. an cell producing, antibodies capable of binding to the FK778 substance) are, for example, spleen cells, lymph node cells and peripheral lymphocytes of an animal (e.g. mouse, rat, rabbit, goat) immunized with the immunogenic substance having increased immunogenicity (e.g. BSA-FR270531 substance conjugate). Antibody-producing cells obtained by allowing the immunogen to act, in a culture medium, on the above-mentioned cells or lymphocytes or the like isolated in advance from the unimmunized animals may also be used. When the latter procedure is used, it is also possible to prepare human-derived antibody-producing cells. The antibody-producing cells and myeloma cells, if they are fusible, may be of different animal species origins but is preferably of the same animal species origin.

The monoclonal antibody production using the cell fusion technique is performed by a conventional method, for example by the principal method of Köhler and Milstein [Nature, 256, 495 (1975)].

In a particularly preferred embodiment, hybridomas are produced by cell fusion between spleen cells obtained from a mouse immunized with a BSA-FR270531 substance conjugate and mouse myeloma cells and screened to afford hybridomas producing a monoclonal antibody specific to the FK778 substance. The said hybridoma is grown in peritoneal cavities of mice and the monoclonal antibody capable of binding to the FK778 substance is obtained from the ascitic fluid of the mice.

(II) Highly-sensitive and Practical Immunoassay Method Utilizing an Antibody for the FK778 Substance

In various immunological assays, antibodies of the present invention capable of binding to the FK778 substance can be used to detect the FK778 substance in a sample in a simple and easy manner with good sensitivity. Such immunological assays include competitive methods (direct method and in direct method), sandwich method, immunoassay with automated analyzers such as ARCHITECT (Abbott Laboratory) and AxSYN (Abbott Laboratory), RIA, ELISA, chemiluminescent immunoassay and so on, all of which are known in the art.

The following methods are examples of a method for assaying the FK778 substance in a sample, and the present invention is not intended to be limited by the following methods.

(i) Competitive Method (Direct-method)

The direct immunoassay method is carried out by immobilizing an antibody capable of binding to the FK778 substance, allowing the FK778 substance contained in a sample and a FK778 substance labeled by detectable substance to react competitively with the said immobilized antibody and detecting the labeled FK778 substance bound to the immobilized antibody.

The said antibody capable of binding to the FK778 substance is the one described in the first aspect (I) of the invention. Both of a polyclonal antibody and a Monoclonal antibody can be used, but a monoclonal antibody is more preferable because it has a high specificity and there are no differences in their specificities between production lots. Usable materials as the solid phase for immobilization are, for example, plates (plates for immunological use, etc.), beads (beads for immunological use, etc.), magnetic microparticles, polystyrene balls, and test tubes. From the simple operation viewpoint, immunological plates and magnetic microparticles are preferred.

Examples of detectable substances for labeling the FK778 substance include various substances known to those skilled in the art such as various enzymes, fluorescent materials, luminescent materials, and radioactive-materials. The suitable enzymes include, for example, peroxidase, β-D-galactosidase, alkaline phosphatase, glucose oxidase, acetylcholine esterase, glucose-6-phosphate dehydrogenase, malate dehydrogenase and urease. Among them, peroxidase (hereinafter referred to as POD) is a preferred enzyme. The suitable fluorescent materials include, for example, fluorescein and fluorescein isothiocyanate. The suitable luminescent materials include acridinium, 1, 2-dioxtetane, luminal and derivative thereof. Acridinium and derivative thereof are preferred.

The detectable substance maybe coupled or conjugated either directly to the FK778 substance or indirectly, through an intermediate (such as, for example, a linker known in the art) using techniques known in the art.

For example, the enzyme-labeled FK778 substance can be prepared by a conventional method. For instance, when a coupling agent is used, the half ester of the FK778 substance with a dicarboxylic acid such as succinic acid as described above in illustrating the first aspect (I) of the invention is reacted with N- hydroxysuccinimide or the like and the resultant activated ester of the said half ester is reacted with an enzyme usable for labeling purposes, for example POD. The enzyme-labeled substance bound to the immobilized antibody can be detected by measuring the activity of the enzyme in a conventional manner. When the enzyme used as the label is POD, the POD bound to the immobilized antibody can be assayed by using an enzyme substrate solution of O-phenylenediamine and hydrogen peroxide and measuring the degree of coloration due to oxidation of the substrate as an optical density. The degree of coloration is proportional to the quantity of the POD-labeled FK778 substance bound to the immobilized antibody.

Alternatively, the luminescence chemistries, such as acridinium ester and acridinium (N-sulfonyl) carboxamide labels, are labeled to FK778 substance. The luminescence chemistries bound to the immobilized antibody is detected by measuring the chemiluminescence in a conventional manner. The method can be used for immunoassay with automated analyzers such as ARCHITECT (Abbott Laboratory).

This direct method can quantitatively,and qualitatively assay very low concentration of the FK778 substance in a simple and easy manner.

(ii) Competitive Method (Indirect-method)

The indirect immunoassay method is performed by using a first antibody capable of binding to a test substance (e.g., FK778 substance) to be assayed and an immobilized second antibody capable of binding to the said first antibody, allowing the test substance contained in a sample and an the same test substance labeled by a detectable substance to react competitively with the said first antibody and detecting the labeled test substance bound to the first antibody bound in turn to the second antibody.

The said indirect method can assay various substances, such as peptides, steroids, prostaglandins, polysaccharides and macrocyclic compounds and is particularly useful in concentration determination of macrocyclic compounds, more specifically the FK778 substance.

The first antibody may be a polyclonal antibody or a monoclonal antibody provided that it can bind to the test substance, but preferably a monoclonal antibody because it has a high specificity and there are no differences in their specificities between production lots. The said first antibody is prepared in the same manner as described in the first aspect (I) of the invention. When the test substance is the FK778 substance, the antibody described above in the first aspect (I) of the invention is useful.

Usable as the second antibody capable of binding to the said first antibody is an antibody prepared by a conventional method using the first antibody, an antibody of the same species as the first antibody as an immunogen or an antibody which is commercially available as well. Any of them, either polyclonal or monoclonal antibody, can be used provided that it will not interfere with the antigen-antibody reaction between the first antibody and the test substance but can bind to the first antibody. When the first antibody is a class IgG antibody obtained from the rabbit, the use of goat anti-rabbit IgG as the second antibody is preferred. When the first antibody is a class IgG antibody obtained, from the mouse, the use of rabbit anti-mouse IgG is preferred.

Alternatively, the indirect immunoassay method is performed by using a first antibody labeled by a first detectable substance, which is capable of binding to a test substance (e.g., FK778 substance) to be assayed, a test substance labeled by a second detectable substance, and an immobilized second antibody capable of binding to said second detectable substance (e.g., ARCHITECT assay). In this case, the amount of the test substance in a sample can be determined by allowing the test substance contained in a sample and an the same test substance labeled by the second detectable substance to react competitively with the said first antibody and detecting the labeled first antibody bound to the labeled test substance whose the second detecting substance is bound to the immobilized second antibody.

The solid phase for immobilization, the detectable substance for labeling the test substance or the first antibody and the method of detecting the said labeling substance are the same as those in the direct method (i) described above. In preferred embodiment, the first detectable substance is acridinium, the second detectable substance is fluorescein, and the second antibody is an anti-FITC antibody.

When this indirect method is employed, the detection limit for test substances can be varied by adjusting the quantity of the first antibody to the quantity of the immobilized second antibody. Thus, very low concentration of the FK778 substance can quantitatively and qualitatively be assayed with high sensitivity and in a simple and easy manner.

(III) Test Kit

The test kit of the present invention is one for the detection of the FK778 substance which comprises an antibody capable of binding to the FK778 substance and the FK778 substance labeled by a detectable substance.

The “antibody capable of binding to the FK778 substance” is either a polyclonal antibody or a monoclonal antibody described above in the first aspect (I) of the invention, but preferably a monoclonal antibody. The said antibody can be supplied in a solid state or in solution.

The “FK778 substance labeled by a detectable substance” is the substance described above. This labeled FK778 substance can also be supplied in a solid state or in solution.

The test kit of the present invention may comprise other ingredients usable when practicing the present highly sensitive immunoassay. For example, the other ingredients include a known quantity of the FK778 substance as a standard for quantitative measurements, an antibody capable of binding to the FK778 antibody and an antibody capable of binding to the detectable substance labeling the FK778 substance. When the detectable substance labeling the FK778 substance is an enzyme, the kit of the present invention may further comprise a substrate for the enzyme.

### EXAMPLE

Methods

Synthesis of FK778 Derivatives Used as an Immunogen

To produce an antibody capable of binding to the FK778 substance, the following four FK778 derivatives used as an immunogen were synthesized. The synthesis scheme for each derivative is shown as follow.

### 1) Synthesis scheme for pentafluorophenyl 6-(4-{[(2Z)-2-cyano-3-hydroxy-2-hepten-6-ynoyl]amino}phenoxy)hexanoate (FR270531)

To a mixture of 6-(4-{[(2Z)-2-cyano-3-hydroxy-2-hepten-6-ynoyl] amino}phenoxy) hexanoic acid (50 mg) (regarding synthesis scheme, see below), C₆F₅OH (37 mg) and 1,4-dioxane (1 mL) was added 1,3-dicyclohexylcarbodiimide (41 mg). The mixture was stirred at ambient temperature overnight.

The mixture was diluted with CHCl₃ and purified by column chromatography on silica gel (elution; 25:1 CHCl₃-MeOH) to afford the product. The product was suspended in diisopropyl ether (4 mL), sonicated and filtered to give pentafluorophenyl 6-(4-{[(2Z)-2-cyano-3-hydroxy-2-hepten-6-ynoyl]amino}phenoxy) hexanoate (68 mg, 93%).

### 2) Synthesis scheme for 6- (4-{ [(2Z) -2-cyano-3-hydroxy-2-hepten-6-ynoyl] amino}phenoxy)hexanoic acid (FR267471)

### i) Preparation for ethyl 6-(4-nitrophenoxy)hexanoate

A mixture of ethyl 6-bromohexanoate (Tokyo kasei Kogyo Co., Ltd.) (5.0 g), 4-nitrophenol (3.43 g), K₂CO₃ (3.41 g) and DMF (25 mL) was stirred at 60° C. for 4 hours.

After cooling, the mixture was partitioned between EtOAc and water. The organic layer was separated, washed successively with 1 NNaOH (three times), water and brine, dried over anhydrous MgSO₄, and concentrated in vacuo. The resulting precipitation was suspended in n-hexane (50mL) and the suspension was sonicated, cooled in an ice-bath and filtered to give ethyl 6-(4-nitrophenoxy)hexanoate (5.7 g, 90%).

### ii) Preparation of ethyl 6-(4-aminophenoxy)hexanoate

A mixture of ethyl 6-(4-nitrophenoxy)hexanoate (5.5 g), 10% Pd/C (50% wet; 0.55 g), EtOH (55 mL) and THF (55 mL) was stirred under 1 atm of H₂ at ambient temperature for 3 hours.

The catalyst was filtered off and the filtrate was concentrated in vacuo. The residue was purified by column chromatography on silica gel (elution; 100:1 CHCl₃-MeOH) to give ethyl 6-(4-aminophenoxy)hexanoate (4.0 g, 81% ).

### iii) Preparation of ethyl 6-{4-[(cyanoacetyl)amino]phenoxy}hexanoate

Cyanoacetic acid (2.0 g) was activated with PCl₅ (5.09 g) in toluene (24 mL). To a solution of activated acid was added ethyl 6-(4-aminophenoxy)hexanoate (4.1 g) and Et₃N (1.64 g) and the mixture was stirred at ambient temperature for an hour.

The mixture was poured into water and extracted with EtOAc. The organic layer was separated, washed with brine, dried over anhydrous Na₂SO₄ and concentrated in vacuo. The residue was purified by column chromatography on silica gel (gradient elution; CHCl₃-MeOH 100:1 to 50:1) to afford the product which was recrystallized from a solvent mixture of EtOAc (4 mL) and diisopropyl ether (1 mL) to give ethyl 6-{4-[(cyanoacetyl)amino]phenoxy}hexanoate.(0.28 g, 39%).

### iv) Preparation of ethyl6-(4-{[(2Z)-2-cyano-3-hydroxy-2-hepten-6-ynoyl]amino}phenoxy)hexanoate

A mixture of ethyl 6-{4-[(cyanoacetyl)amino]phenoxy}hexanoate (2.2 g), HO₂C(CH₂)₂C≡CH (813 mg), K₂CO₃ (2.29 g) and THF (18 mL) was stirred at 50° C. for half an hour. To the mixture was added dropwise a solution of ClCO₂ⁱPr (1.19 g) in THF (4.4 mL).

The mixture was poured into water and extracted twice with EtOAc. The organic layer was combined, washed with brine, dried over anhydrous Na₂SO₄ and concentrated in vacuo. The residue was purified by column chromatography on silica gel (gradient elution; CHCl₃-1%NHOH/MeOH 50:1 to 20:1 to 10:1) to give ethyl 6-(4-{[(2Z)-2-cyano-3-hydroxy-2-hepten-6-ynoyl]amino}phenoxy)hexanoate (0.81 g, 29 %).

### v) Preparation of 6-(4-{[(2Z)-2-cyano-3-hydroxy-2-hepten -6-ynoyl]amino}phenoxy)hexanoic acid

To a mixture of ethyl 6-(4-{[(2Z)-2-cyano-3-hydroxy-2-hepten -6-ynoyl]amino}phenoxy)hexanoate (0.80 g) and EtOH (2 mL) was added a solution of 4 N NaOH (2 mL). The mixture was stirred at ambient temperature overnight.

The mixture was concentrated in vacuo and the residue was dissolved in water (20 mL). The solution was cooled in an ice-bath and acidified with concentrated HCl (1 mL). The resulting suspension was diluted with water (25 mL) and stirred at ambient temperature for half an hour. The precipitate was collected, dried in vacuo and recrystallized from EtOH (10 mL) to give 6-(4-{[(2Z)-2-cyano-3-hydroxy-2-hepten-6-ynoyl]amino}phenoxy)hexanoic acid (0.48 g, 64%).

### 3) Synthesis scheme for (5Z)-6-cyano-5-hydroxy-7-oxo-7-{ [4-(trifluoromethyl)phenyl]amino}-5-heptenoic acid (FR266831)

### i) Preparation of 2-cyano-N-[4-(trifluoromethyl) phenyl]acetamide

Cyanoacetic acid (76.5 g) was activated with PCl₅ (194.3 g) in toluene (900 mL). To a solution of activated acid was added [4- (trifluoromethyl)phenyl] amine (Tokyo kasei Kogyo Co., Ltd.) (100 g) and Et₃N (62.7 g). To the reaction mixture was added water (600 mL) to crystallize 2-cyano-N-[4-(trifluoromethyl) phenyl]acetamide. The precipitate was collected by filtration, washed with water (100 mL) and methanol (50 ml), and then dried in vacuo. (121 g, 86%)

### ii) Preparation of ethyl (5Z)-6-cyano-5-hydroxy-7-oxo-7-{[4-(trifluoromethyl)phenyl]amino}-5-heptenoate

To a mixture of 2-cyano-N-[4-(trifluoromethyl) phenyl]acetamide (2.0g) and THF (80mL) was added NaH (771 mg) portionwise below 10° C. The mixture was stirred at ambient temperature for 2.5 hours. To the mixture was added a solution of ClCO(CH₂)₃CO₂Et (1.88 g) in THF (8 mL) dropwise while the internal temperature rose to 30° C. After addition, the mixture was stirred at ambient temperature for an hour.

The mixture was poured into water and extracted twice with EtOAc. The organic layers were combined, washed with brine, dried over anhydrous Na₂SO₄ and concentrated in vacuo. The residue was purified by column chromatography on silica gel (gradient elution; 25:1 CHCl₃-MeOH to 100:4:1 CHCl₃-MeOH—HCO₂H) to afford the product which was recrystallized from EtOH (20 mL) to give ethyl (5Z)-6-cyano-5-hydroxy-7-oxo-7-{[4-(trifluoromethyl)phenyl]amino}-5-heptenoate (0.95 g, 29%).

### iii) Preparation of (5Z)-6-cyano-5-hydroxy-7-oxo-7-{[4-(trifluoromethyl)phenyl]amino}-5-heptenoic acid

To a mixture of ethyl (5Z)-6-cyano-5-hydroxy-7-oxo-7-{ [4-(trifluoromethyl)phenyl]amino}-5-heptenoate (0.95 g) and EtOH (2.5 mL) was added a solution of 4 N NaOH (4 mL). The mixture was stirred at ambient temperature for 10 minutes.

The mixture was concentrated in vacuo and the residue was dissolved in water (10 mL). The solution was cooled in an ice-bath and acidified with concentrated HCl (3 mL). The resulting suspension was diluted with water (20 mL) and stirred at ambient temperature for half an hour. The precipitate was collected, dried in vacuo and recrystallized from EtOH to give (5Z)-6-cyano-5-hydroxy-7-oxo-7-{[4-(trifluoromethyl)phenyl]amino}-5-heptenoic acid (0.75 g, 85%).

### 4) Synthesis scheme for (2Z)-2-cyano-3,5-dihydroxy-N-[4-(trifluoromethyl)phenyl]-2-hepten-6-ynamide (FR271764)

### i) Preparation of 2-cyano-3-oxo-N-[4-(trifluoromethyl)phenyl]butanamide

A mixture of 2-cyano-N-[4-(trifluoromethyl) phenyl]acetamide (70 g), AcOH (22.11 g), K₂CO₃ (101.76 g) and THF (560 mL) was stirred at 50° C. for half an hour. To the mixture was added dropwise a solution of ClCO₂ⁱPr (52.64 g) in THF (70 mL).

After cooling, water (420 mL) was added to the mixture. The mixture was acidified by the addition of 17.5% HCl (210 mL). The mixture was added with PhMe (315 mL) and stirred for 15 minutes at ambient temperature. The resulting precipitation was collected and dried to give 2-cyano-3-oxo-N- [4- (trifluoromethyl)phenyl]butanamide (52.4 g, 63%).

### ii) Preparation of (2Z)-2-cyano-3,5-dihydroxy-N-[4-(trifluoromethyl)phenyl]-7-(trimethylsilyl)-2-hepten-6-ynamide

A 2.4 M solution of n-BuLi in hexanes (25 mL) was cooled to −50° C. To the solution was added a solution of 2-cyano-3-oxo-N -[4-(trifluoromethyl)phenyl]butanamide (5.0 g) in THF (200 mL) dropwise over half an hour while maintaining the internal temperature around −50° C. After addition, the mixture was stirred at −50° C. for half an hour. To the mixture was added a solution of OHC—C≡C-TMS (2.34 g) in THF (5 mL) dropwise over half an hour while the internal temperature was maintained around −50° C. After addition, the mixture was allowed to stir for half an hour at which time the internal temperature came to −30° C.

The reaction mixture was transferred into a dropping funnel and added dropwise to a cold solution of 1 M citric acid (120 mL) below 10° C. (The pH of the mixture became 3.5.)

The mixture was extracted once with EtOAc, and the extract was washed with brine, dried over anhydrous Na₂SO₄ and concentrated in. vacuo. The residue was purified by column chromatography on silica gel (gradient elution; n-hexane-acetone 2:1 to 1:1) to give (2Z)-2-cyano-3,5-dihydroxy-N-[4-(trifluoromethyl) phenyl]-7-(trimethylsilyl)-2-hepten-6-ynamide (2.8 g, 38%).

### iii) Preparation of (2Z)-2-cyano-3,5-dihydroxy-N-[4-(trifluoromethyl)phenyl]-2-hepten-6-ynamide

To a solution of (2Z)-2-cyano-3,5-dihydroxy-N-[4-(trifluoromethyl)phenyl]-7-(trimethylsilyl)-2-hepten-6-yn amide (2.3 g) in MeOH (69 mL) was added K₂CO₃ (4.81 g) and the mixture was stirred at ambient temperature for 40 minutes.

The reaction mixture was concentrated in vacuo and the residue was added with 1 M citric acid (70 mL). The mixture was extracted once with EtOAc, and the extract was washed with brine, dried over an hydrous Na₂SO₄ and concentrated in vacuo. The residue was purified by column chromatography on silica gel (gradient elution; n-hexane-acetone 2:1 to 3:2) to afford the product. The product was dissolved in hot EtOH (4 mL) and diluted with diisopropyl ether (8 mL) with stirring. After cooled to ambient temperature, the mixture was diluted with additional diisopropyl ether (4 mL) and aged in an ice-bath. The suspension was filtered to give (2Z)-2-cyano-3,5-dihydroxy-N-[4-(trifluoromethyl) phenyl]-2-hepten-6-ynamide (0.51 g, 27%).

Selection of an Immunogen to Obtain the High Titer of Antibody Against the FK778 Substance

To obtain the selective antibody against FK778, two kinds of immunogens which were conjugated (FR267471 and FR266831) with bovine thyroglobulin (Sigma-Aldrich Corp.) mixed with Freund's complete adjuvant (FCA) (Difco) were immunized in hyperimmune Balb/c mice. After four times immunizations, the titer of antibody against the FR267471-BSA or the FR266831-BSA in sera was measured by enzyme-linked immunosorbent assay. Unfortunately, It was considered that the titer for both immunogens were not sufficient for establish the selective immunoassay method against FK778. To obtain immunogens having higher titer than the two immunogens, another type of immunogen FR270531 was synthesized. After seven immunize with same way, the titer from FR270531 was improved and was higher than those of FR267471 and FR266831. Accordingly, the present inventors have used FR270531 as an immunogen to produce antibodies directed to FK778, and FR267471 and FR266831 as positive and negative control to select the FK778 antibody specifically binding to the FK778 substance.

1. Preparation of Immunogen

FR270531 was dissolved in N,N-dimethylformamide at a concentration of 21 mg/mL. Bovine thyroglobulin was dissolved in 0. 01 mol/L phosphate buffer (pH6.0) at a concentration of 5 mg/mL. 300 μL of the FR270531 solution was mixed with 1.76 mL of the bovine thyroglobulin solution, and then stirred at room temperature for 1 h. Then, the mixture was dialyzed against PBS, and used as immunogen.

2. Preparation of Conjugates for Hybridoma Screening

Bovine serum albumin (BSA) (Sigma-Aldrich Corp.) dissolved in 0. 01 mol/L phosphate buffer (pH6.0) at a concentration of 5 mg/mL was used as carrier protein. FR267471 and FR266831 were dissolved in N,N-dimethylformamide at a concentration of 21 mg/mL and 17 mg/mL, respectively. Bovine thyroglobulin was dissolved in 0. 01 mol/L phosphate buffer (pH6.0) at a concentration of 5 mg/mL. 48 μL of the FR267471 solution was mixed with 82 μL of BSA solution, then was stirred at room temperature for 1 h. The mixture was then dialyzed against PBS, and used as antigen for ELISA.

100 μL of the FR266831 solution was mixed with 31 μL of DCC solution (100mg/mL solution of N,N-dicyclohexyl carbodiimide in N,N-dimethylformamide) (Wako Pure Chemical Industries, Ltd.) at a molar ratio of 1:3, and then stirred at room temperature for 30 min. The mixture was then mixed with 17 μL of NHS solution (100 mg/mL solution of N,N-Hydroxysuccinimide in N,N-dimethylformamide) (Wako Pure Chemical Industries, Ltd.) at a molar ratio of 1:3 and stirred at room temperature, for 1 h. The pH of the mixture was adjusted to 3.0 with 60 μL of 0.1 N HCl. After 1 h stirring at room temperature, the pH of the mixture was adjusted to 6.0-7.0 with 110 μL of 0.1N NaOH. Then, 318 μL of the mixture was mixed with 650 μL of BSA solution at a molar ratio of 100:1, and stirred at room temperature for 3 h. The mixture was dialyzed against PBS, and used as antigen for ELISA.

3. Monoclonal Antibody Production

1) Immunization

Immunogen FR270531 prepared as described above was mixed with Freund's complete adjuvant, then 50 μg/mouse of immunogen was injected into 4 mice (BALB/c), once a week, subcutaneously. After 5immunizations, blood samples were collected and the titer of antibody against FR267471-BSA or FR266831-BSA in sera was measured by antigen-coated enzyme-linked immunosorbent assay (ELISA), described in detail below (Table 1). Immunization was repeated 2 more times, then blood samples were collected and the titer of antibody measured again (Table 2).

2) Fusion

After a single booster, spleen cells (8.5×10⁸ cells) were collected and fused with X63-Ag8-653 cells by the polyethylene glycol-mediated cell fusion technique, and seeded to thirty-three 96-well plates.

3) Screening Hybridomas

After 9 days culture at 37° C., the hybridoma cells were screened with ELISA using FR267471-BSA as a positive plate and FR266831-BSA as a negative plate. Cells from 49 wells produced FR267471 specific mAbs (designated as No.1-49 in Table 3) and cells from 85 wells produced mabs against both FR267471 and FR266831 (designated as No. 50-134 in Table 3).

These antibodies were tested for their cross reactivities for BSA. Cells from 49 wells produced FR267471 specific mAbs (designated as No.1-49 in Table 4) and cells from 11 wells had cross-reactive mAbs against both FR267471 and FR266831 (designated as No. 54, 55, 57, 65, 72, 81, 83, 108, 118, 122 and 132 in Table 4).

After 5 days culture, antibodies were tested to determine if their reactivity to FR267471-BSA was competitive with exogenously added FK778, and 8 clones were selected (No. 7, 9, 14, 18, 20, 24, 28, 31, Table 5).

4) Limiting Dilution

Cells were cloned by limiting dilution, followed by ELISA screening. 3 clones (e.g., designated as No. 7A, 7B and 7C) were selected for each clone, No. 7, 9, 14, 18, 20, 24, 28, 31 (total 24 clones in Table 6). Then, each No. A clone was subcloned. 3 clones were selected from the No. A subclones (e.g., designated as No. 7A1, 7A2, and 7A3 in Table 7). These were each cultured in 4 wells of 24-well plate, frozen and stored.

Measurement of Titer Against Immunogen in Immunized Sera Using Antigen-coated Enzyme-linked Immunosorbent Assay (ELISA)

Microtiter plates (96 well; Greiner) were coated with FR267471-BSA or FR266831-BSA (50 μL per each well; 1 μg/mL in 0.1M carbonate buffer, pH9.5) at 4° C. overnight, then blocked with 200 μL of 0.1% bovine serum albumin (BSA) in PBS, containing 0.05% NaN₃ (referred to as blocking buffer). Antisera were diluted serially with dilution buffer (1% BSA in PBS, containing 0.05% Tween-20), then added to the antigen coated 96-well plates. After incubation for 30 min at 37° C., each well was washed with washing buffer (0.05% Tween-20 in 10 mM phosphate buffer, pH7.5). 50 μL of 125 ng/mL horseradish peroxidase-labeled anti-mouse IgG (H+L) goat IgG Fab′ antibody (IBL) was added to each well and incubated for 30 min at 37° C. After washing with washing buffer, 100 μL of 400 ,μg/mL o-PD (o-Phenylenediamine, Sigma) in K₂HPO₄-citrate buffer (pH5.1) (substrate buffer) was added to each well and incubated for 15 min at room temperature in the dark. Color development was stopped by addition of 100 μL of stop solution (1N H₂SO₄). Optical density (OD) at 490 nm for each well was measured. The titer of measured antisera was defined as the dilution rate which showed more than 0.2 OD at 490 nm.

Protocol for Competition Test of Each Hybridoma Using FK778

50 μL of culture supernatant from each hybridoma was mixed with or without 50 μL of FK778 or FR271764 solution for the determination of cross reactivity, then incubated overnight at 4° C. 50 μL of the mixture was added to the FR267471-BSA plate, then incubated for 30 min at 37° C. 50 μL of anti-Mouse IgG Goat Fab′-HRP conjugate was added and incubated for 30 min at 37° C. 100 μL of 400 μg/mL o-PD (o-Phenylenediamine, Sigma) in K₂HPO₄-citrate buffer (pH5.1) (substrate) was added to each well and incubated for 15 min at room temperature in the dark. Color development was stopped by addition of 100 μL of stop solution (1N H₂SO₄). OD at 490 nm for each well was measured.

Results

1. Titers of Sera After 5 and 7 Immunizations

After 5 immunizations, antisera were collected and the titer of antibody measured. 4 mice showed x6400 titer (Table 1). After an additional two immunizations, antiserum titers raised to x12800 or x25600 (Table 2).

TABLE 1
Titers of sera after 5 immunizations to FR267471 or FR266831

| Mice No. | Dilution rate of sera | | | | | | | | | | | |
|---|---|---|---|---|---|---|---|---|---|---|---|---|
| ID | ×100 | ×200 | ×400 | ×800 | ×1600 | ×3200 | ×6400 | ×12800 | ×25600 | ×51200 | ×102400 | Blank |

1) FR267471-BSA coated plate

| | | | | | | | | | | | | |
|---|---|---|---|---|---|---|---|---|---|---|---|---|
| No. 1 | 1.74 | 1.45 | 1.07 | 0.88 | 0.64 | 0.45 | 0.25 | 0.16 | 0.11 | 0.06 | 0.03 | 0.00 |
| Right | 1.70 | 1.37 | 1.06 | 0.78 | 0.60 | 0.39 | 0.23 | 0.13 | 0.08 | 0.04 | 0.02 | 0.00 |
| No. 2 | 1.67 | 1.31 | 1.00 | 0.71 | 0.51 | 0.33 | 0.19 | 0.10 | 0.06 | 0.02 | 0.01 | 0.00 |
| Left | 1.69 | 1.26 | 0.99 | 0.69 | 0.49 | 0.31 | 0.18 | 0.10 | 0.06 | 0.02 | 0.01 | 0.00 |
| No. 3 | 1.73 | 1.34 | 1.11 | 0.81 | 0.59 | 0.39 | 0.24 | 0.13 | 0.07 | 0.03 | 0.02 | 0.00 |
| Both | 1.68 | 1.39 | 1.12 | 0.81 | 0.58 | 0.40 | 0.22 | 0.13 | 0.08 | 0.03 | 0.02 | 0.00 |
| No. 4 | 1.62 | 1.34 | 1.04 | 0.77 | 0.54 | 0.35 | 0.21 | 0.11 | 0.06 | 0.03 | 0.02 | 0.00 |
| None | 1.70 | 1.39 | 1.11 | 0.79 | 0.57 | 0.39 | 0.23 | 0.13 | 0.07 | 0.03 | 0.02 | 0.00 |

2) FR266831-BSA coated plate

| | | | | | | | | | | | | |
|---|---|---|---|---|---|---|---|---|---|---|---|---|
| No. 1 | 0.40 | 0.22 | 0.10 | 0.06 | 0.04 | 0.03 | 0.01 | 0.00 | 0.01 | 0.01 | 0.00 | 0.01 |
| Right | 0.33 | 0.19 | 0.09 | 0.04 | 0.02 | 0.01 | 0.00 | 0.00 | 0.00 | 0.00 | 0.00 | 0.00 |
| No. 2 | 0.31 | 0.14 | 0.07 | 0.03 | 0.02 | 0.01 | 0.00 | 0.00 | 0.00 | 0.00 | 0.00 | 0.00 |
| Left | 0.33 | 0.15 | 0.06 | 0.03 | 0.01 | 0.00 | 0.00 | 0.00 | 0.00 | 0.00 | 0.00 | 0.00 |
| No. 3 | 0.31 | 0.14 | 0.07 | 0.03 | 0.01 | 0.00 | 0.00 | 0.00 | 0.00 | 0.00 | 0.00 | 0.00 |
| Both | 0.27 | 0.14 | 0.08 | 0.03 | 0.02 | 0.01 | 0.00 | 0.00 | 0.00 | 0.00 | 0.00 | 0.00 |
| No. 4 | 0.28 | 0.17 | 0.08 | 0.04 | 0.02 | 0.01 | 0.01 | 0.00 | 0.00 | 0.00 | 0.00 | 0.00 |
| None | 0.41 | 0.17 | 0.09 | 0.05 | 0.02 | 0.01 | 0.01 | 0.00 | 0.00 | 0.00 | 0.00 | 0.00 |

Dilution rates showing more than 0.2 OD are underlined.

TABLE 2
Titers of sera after 7 immunizations to FR267471 or FR266831

| Mice No. | Dilution rate of Sera | | | | | | | | | | | |
|---|---|---|---|---|---|---|---|---|---|---|---|---|
| ID | ×100 | ×200 | ×400 | ×800 | ×1600 | ×3200 | ×6400 | ×12800 | ×25600 | ×51200 | ×102400 | Blank |

1) FR267471-BSA coated plate

| No. 1 | 2.22 | 2.03 | 1.78 | 1.41 | 1.11 | 0.80 | 0.56 | 0.33 | 0.22 | 0.12 | 0.07 | 0.01 |
|---|---|---|---|---|---|---|---|---|---|---|---|---|
| Right | 2.29 | 2.10 | 1.87 | 1.52 | 1.12 | 0.83 | 0.53 | 0.33 | 0.19 | 0.12 | 0.08 | 0.00 |
| No. 2 | 2.17 | 1.94 | 1.70 | 1.42 | 1.02 | 0.76 | 0.51 | 0.31 | 0.19 | 0.11 | 0.06 | 0.00 |
| Left | 2.14 | 1.90 | 1.64 | 1.38 | 0.98 | 0.73 | 0.48 | 0.29 | 0.17 | 0.10 | 0.06 | 0.00 |
| No. 3 | 1.89 | 1.67 | 1.45 | 1.19 | 0.87 | 0.62 | 0.40 | 0.25 | 0.15 | 0.08 | 0.05 | 0.00 |
| Both | 1.80 | 1.69 | 1.41 | 1.24 | 0.87 | 0.62 | 0.40 | 0.24 | 0.14 | 0.08 | 0.04 | 0.00 |
| No. 4 | 2.03 | 1.96 | 1.81 | 1.43 | 1.14 | 0.85 | 0.58 | 0.36 | 0.21 | 0.13 | 0.07 | 0.00 |
| None | 2.15 | 1.93 | 1.74 | 1.44 | 1.10 | 0.83 | 0.54 | 0.34 | 0.20 | 0.13 | 0.07 | 0.00 |
| Mice No. | Dilution rate of Sera | | | | | | | | | | | |
| ID | ×100 | ×200 | ×400 | ×800 | ×1600 | ×3200 | ×6400 | ×12800 | ×25600 | ×51200 | ×102400 | Blank |

2) FR266831-BSA coated plate

| | | | | | | | | | | | | |
|---|---|---|---|---|---|---|---|---|---|---|---|---|
| No. 1 | 0.27 | 0.16 | 0.10 | 0.06 | 0.04 | 0.02 | 0.01 | 0.01 | 0.01 | 0.00 | 0.00 | 0.00 |
| Right | 0.23 | 0.15 | 0.09 | 0.05 | 0.03 | 0.01 | 0.00 | 0.00 | 0.00 | 0.00 | 0.00 | 0.00 |
| No. 2 | 0.41 | 0.22 | 0.12 | 0.07 | 0.03 | 0.02 | 0.01 | 0.00 | 0.00 | 0.00 | 0.00 | 0.00 |
| Left | 0.41 | 0.21 | 0.13 | 0.06 | 0.03 | 0.01 | 0.01 | 0.00 | 0.00 | 0.00 | 0.00 | 0.00 |
| No. 3 | 0.25 | 0.12 | 0.08 | 0.04 | 0.02 | 0.01 | 0.00 | 0,00 | 0.00 | 0.00 | 0.00 | 0.00 |
| Both | 0.25 | 0.14 | 0.08 | 0.04 | 0.02 | 0.01 | 0.00 | 0.00 | 0.00 | 0.00 | 0.00 | 0.00 |
| No. 4 | 0.24 | 0.15 | 0.09 | 0.05 | 0.03 | 0.01 | 0.01 | 0.00 | 0.00 | 0.00 | 0.00 | 0.00 |
| None | 0.27 | 0.15 | 0.10 | 0.06 | 0.03 | 0.02 | 0.01 | 0.00 | 0.00 | 0.00 | 0.00 | 0.00 |

Dilution rates showing more than 0.2 OD are underlined.
Right and None mice were used for fusion.

2. First Screening of Hybridomas

Hybridoma cells were screened with ELISA using FR267471-BSA as a positive plate and FR266831-BSA as a negative plate. Cells from 49 wells produced FR267471 specific mabs (No. 1-49) and cells from 85 wells produced mAbs against both FR267471 and FR266831 (No. 50-134) (Table 3).

TABLE 3
OD value at 490 nm of first screening for hybridoma producing
mAbs against FR267471 or both FR267471 and FR266831

| Cell No. | Pos | Neg |
|---|---|---|
| 1 | 1.160 | 0.073 |
| 2 | 1.015 | 0.060 |
| 3 | 1.216 | 0.005 |
| 4 | 1.866 | 0.034 |
| 5 | 1.882 | −0.005 |
| 6 | 2.237 | 0.122 |
| 7 | 2.852 | 0.113 |
| 8 | 2.079 | 0.103 |
| 9 | 2.587 | 0.086 |
| 10 | 1.619 | 0.107 |
| 11 | 1.194 | 0.063 |
| 12 | 1.964 | 0.038 |
| 13 | 1.268 | 0.073 |
| 14 | 2.559 | 0.025 |
| 15 | 2.922 | 0.003 |
| 16 | 1.117 | 0.055 |
| 17 | 1.299 | 0.001 |
| 18 | 1.499 | 0.025 |
| 19 | 1.308 | 0.061 |
| 20 | RO | 0.040 |
| 21 | 1.161 | 0.048 |
| 22 | 1.092 | 0.188 |
| 23 | 1.055 | 0.039 |
| 24 | RO | 0.195 |
| 25 | 1.328 | 0.163 |
| 26 | 1.332 | 0.024 |
| 27 | 1.268 | 0.076 |
| 28 | 2.774 | 0.059 |
| 29 | 2.574 | 0.012 |
| 30 | 1.135 | 0.031 |
| 31 | RO | 0.174 |
| 32 | 1.045 | 0.109 |
| 33 | 1.190 | 0.036 |
| 34 | 1.808 | 0.088 |
| 35 | 1.178 | 0.128 |
| 36 | 1.038 | 0.190 |
| 37 | 1.031 | 0.060 |
| 38 | 1.419 | 0.154 |
| 39 | 1.124 | 0.106 |
| 40 | 1.311 | 0.017 |
| 41 | 1.351 | 0.110 |
| 42 | 1.312 | 0.131 |
| 43 | 1.040 | 0.001 |
| 44 | 1.431 | 0.140 |
| 45 | 1.385 | 0.180 |
| 46 | 1.604 | 0.178 |
| 47 | 1.389 | 0.151 |
| 48 | 1.307 | 0.045 |
| 49 | 1.209 | 0.028 |
| 50 | 2.321 | 2.673 |
| 51 | 2.265 | RO |
| 52 | 2.481 | 2.893 |
| 53 | 2.158 | 1.511 |
| 54 | 2.474 | 1.856 |
| 55 | RO | 2.637 |
| 56 | 1.797 | 1.387 |
| 57 | 1.626 | 1.019 |
| 58 | 1.561 | 1.104 |
| 59 | 2.464 | 1.754 |
| 60 | 1.522 | 1.315 |
| 61 | 1.642 | 1.475 |
| 62 | 1.830 | 1.381 |
| 63 | RO | RO |
| 64 | 1.880 | 1.702 |
| 65 | 1.756 | 1.922 |
| 66 | 1.691 | 1.441 |
| 67 | 1.590 | 1.192 |
| 68 | RO | RO |
| 69 | RO | 2.706 |
| 70 | 1.581 | 1.036 |
| 71 | RO | 2.773 |
| 72 | 2.204 | 1.202 |
| 73 | 2.268 | 2.332 |
| 74 | 2.133 | 2.237 |
| 75 | 1.756 | 1.412 |
| 76 | 2.082 | 2.124 |
| 77 | 1.457 | 1.208 |
| 78 | 2.688 | 2.089 |
| 79 | RO | RO |
| 80 | RO | RO |
| 81 | 1.514 | 1.530 |
| 82 | 1.208 | 1.189 |
| 83 | 2.654 | 1.281 |
| 84 | RO | 2.439 |
| 85 | 1.368 | 1.252 |
| 86 | 1.639 | 1.052 |
| 87 | RO | RO |
| 88 | 2.757 | 1.502 |
| 89 | 1.910 | 1.381 |
| 90 | 1.628 | 1.193 |
| 91 | RO | 2.483 |
| 92 | 2.059 | 1.765 |
| 93 | 1.454 | 1.103 |
| 94 | RO | RO |
| 95 | RO | 2.723 |
| 96 | 1.411 | 1.021 |
| 97 | 1.231 | 1.220 |
| 98 | 1.906 | 1.578 |
| 99 | 2.357 | 2.155 |
| 100 | RO | RO |
| 101 | 1.495 | 1.370 |
| 102 | 2.502 | 2.627 |
| 103 | RO | RO |
| 104 | 2.315 | 2.003 |
| 105 | 1.797 | 1.493 |
| 106 | RO | RO |
| 107 | 2.154 | 1.950 |
| 108 | 2.714 | 2.461 |
| 109 | 2.813 | 2.921 |
| 110 | 2.062 | 1.495 |
| 111 | 1.693 | 1.222 |
| 112 | 1.809 | 1.101 |
| 113 | 1.396 | 1.112 |
| 114 | 2.459 | 1.712 |
| 115 | 2.155 | 1.564 |
| 116 | RO | 2.182 |
| 117 | 1.113 | 1.150 |
| 118 | 1.172 | 1.440 |
| 119 | 1.720 | 1.063 |
| 120 | 1.585 | 1.199 |
| 121 | 2.090 | 1.755 |
| 122 | RO | RO |
| 123 | 2.297 | 1.473 |
| 124 | RO | 2.576 |
| 125 | 2.684 | 1.804 |
| 126 | RO | RO |
| 127 | 2.128 | 1.567 |
| 128 | 1.641 | 1.245 |
| 130-1 | 1.945 | 1.071 |
| 130-2 | 2.319 | 1.342 |
| 131-1 | RO | 2.927 |
| 131-2 | 2.161 | 1.254 |
| 132 | 1.887 | 1.432 |
| 133 | 2.119 | 1.537 |
| 134 | 2.092 | 1.616 |

Pos: FK778 (FR267471)-BSA plate
Neg: FK778 (FR266831)-BSA plate
RO: out of range
No. 1-49 hybridoma cells produce FR267471 specific mAbs.
No. 50-134 hybridoma cells produce mAbs against both FR267471 and FR266831.

3. Specificity and Cross-reactivity of Hybridomas

After the first screening, the hybridomas were tested for cross-reactivity to BSA. Cells from 49 wells produced FR267471 specific mAbs (No. 1-49) and cells from 11 wells cross reactive mAbs against both FR267471 and FR266831 (No. 54-132) (Table 4).

TABLE 4
OD value at 490 nm of selected hybridomas
against positive, negative and BSA plate

| Cell No. | Pos | Neg | BSA |
|---|---|---|---|
| 1 | 1.050 | 0.011 | 0.001 |
| 2 | 0.300 | 0.002 | 0.001 |
| 3 | 0.584 | 0.000 | 0.001 |
| 4 | 0.954 | −0.000 | −0.001 |
| 5 | 0.818 | −0.003 | 0.000 |
| 6 | 1.103 | 0.021 | 0.000 |
| 7 | 1.791 | 0.022 | 0.007 |
| 8 | 1.171 | 0.019 | 0.014 |
| 9 | 2.189 | 0.018 | 0.032 |
| 10 | 0.975 | 0.023 | 0.009 |
| 11 | 0.514 | 0.000 | 0.001 |
| 12 | 1.414 | 0.013 | −0.001 |
| 13 | 0.656 | −0.001 | 0.000 |
| 14 | 1.862 | 0.026 | 0.008 |
| 15 | 2.267 | 0.018 | 0.000 |
| 16 | 0.324 | 0.047 | 0.014 |
| 17 | 0.894 | 0.000 | 0.000 |
| 18 | 1.299 | 0.018 | 0.000 |
| 19 | 0.656 | 0.010 | 0.003 |
| 20 | 2.675 | 0.021 | −0.001 |
| 21 | 0.584 | 0.010 | 0.001 |
| 22 | 0.750 | 0.062 | −0.002 |
| 23 | 0.572 | 0.004 | 0.000 |
| 24 | RO | 0.063 | 0.003 |
| 25 | 0.894 | 0.033 | 0.006 |
| 26 | 0.853 | 0.001 | −0.002 |
| 27 | 0.818 | 0.024 | 0.001 |
| 28 | 1.438 | 0.019 | −0.001 |
| 29 | 2.117 | 0.006 | −0.003 |
| 30 | 0.890 | −0.001 | 0.000 |
| 31 | 2.831 | 0.070 | 0.029 |
| 32 | 0.463 | 0.015 | 0.004 |
| 33 | 0.521 | 0.006 | 0.000 |
| 34 | 1.183 | 0.008 | 0.037 |
| 35 | 0.413 | 0.059 | 0.014 |
| 36 | 0.387 | 0.110 | 0.011 |
| 37 | 0.341 | 0.000 | −0.002 |
| 38 | 0.614 | 0.036 | 0.006 |
| 39 | 0.422 | 0.008 | 0.000 |
| 40 | 0.514 | 0.004 | 0.000 |
| 41 | 0.641 | 0.011 | 0.007 |
| 42 | 0.533 | 0.015 | 0.002 |
| 43 | 0.557 | 0.000 | 0.000 |
| 44 | 0.649 | 0.042 | −0.003 |
| 45 | 0.737 | 0.177 | 0.021 |
| 46 | 1.182 | 0.034 | 0.037 |
| 47 | 0.763 | 0.044 | 0.000 |
| 48 | 0.712 | 0.010 | 0.000 |
| 49 | 0.909 | 0.000 | 0.000 |
| 54 | 0.395 | 0.414 | 0.195 |
| 55 | 0.474 | 0.149 | 0.132 |
| 57 | 0.877 | 0.886 | 0.311 |
| 65 | 0.725 | 0.571 | 0.241 |
| 72 | 0.704 | 0.349 | 0.306 |
| 81 | 0.499 | 0.466 | 0.190 |
| 83 | 0.752 | 0.192 | 0.332 |
| 108 | 0.910 | 1.060 | 0.176 |
| 118 | 0.470 | 0.378 | 0.021 |
| 122 | 0.880 | 0.502 | 0.270 |
| 132 | 0.732 | 0.611 | 0.273 |

Pos: FK778 (FR267471)-BSA plate
Neg: FK778 (FR266831)-BSA plate
BSA: BSA plate
RO: out of range
No. 1-49 hybridoma cells produce FR267471 specific mAbs.
No. 54, 55, 57, 65, 72, 81, 83, 108, 118, 122, and 132 hybridoma cells produce mAbs against both FR267471 and FR266831.

Competition Test for Each Hybridoma Using FK778

The antibodies were tested for their reactivity to FR267471-BSA and competition with exogenously added FK778. From this test 8 mAbs were selected (No. 7, 9, 14, 18, 20, 24, 28, 31, Table 5).

TABLE 5
OD value at 490 nm of competition test
for each hybridoma using FK778

FK778

| Cell No. | with | without |
|---|---|---|
| 1 | 0.007 | 0.385 |
| 2 | 0.003 | 0.046 |
| 3 | 0.004 | 0.082 |
| 4 | 0.003 | 0.168 |
| 5 | 0.001 | 0.459 |
| 6 | 0.005 | 0.438 |
| 7 | 0.009 | 2.436 |
| 8 | 0.013 | 0.343 |
| 9 | 0.051 | 2.278 |
| 10 | 0.005 | 0.660 |
| 11 | 0.005 | 0.047 |
| 12 | 0.003 | 0.773 |
| 13 | 0.001 | 0.861 |
| 14 | 0.029 | 2.376 |
| 15 | 0.001 | 0.947 |
| 16 | 0.004 | 0.022 |
| 17 | 0.007 | 0.122 |
| 18 | 0.003 | 1.928 |
| 19 | 0.044 | 0.119 |
| 20 | 0.002 | 2.070 |
| 21 | 0.002 | 0.102 |
| 22 | −0.001 | 0.271 |
| 23 | −0.002 | 0.082 |
| 24 | 0.014 | 3.086 |
| 25 | 0.030 | 0.066 |
| 26 | 0.003 | 0.379 |
| 27 | 0.003 | 0.052 |
| 28 | 0.004 | 2.824 |
| 29 | 0.001 | 0.953 |
| 30 | −0.002 | 0.111 |
| 31 | 0.033 | 2.242 |
| 32 | 0.004 | 0.042 |
| 33 | 0.005 | 0.019 |
| 34 | 0.003 | 0.161 |
| 35 | 0.002 | 0.057 |
| 36 | 0.005 | 0.014 |
| 37 | −0.001 | 0.025 |
| 38 | 0.002 | 0.076 |
| 39 | −0.001 | 0.033 |
| 40 | 0.004 | 0.040 |
| 41 | 0.013 | 0.092 |
| 42 | 0.112 | 0.028 |
| 43 | 0.003 | 0.426 |
| 44 | 0.004 | 0.073 |
| 45 | 0.022 | 0.094 |
| 46 | 0.076 | 0.804 |
| 47 | −0.001 | 0.102 |
| 48 | 0.004 | 0.084 |
| 49 | 0.017 | 0.826 |
| 54 | 0.195 | 0.169 |
| 55 | 0.168 | 0.377 |
| 57 | 0.090 | 0.079 |
| 65 | 0.405 | 0.023 |
| 72 | 0.800 | 0.606 |
| 81 | 0.112 | 0.019 |
| 83 | 0.164 | 0.063 |
| 108 | 0.171 | 0.135 |
| 118 | 0.096 | 0.036 |
| 122 | 0.911 | 0.282 |
| 132 | 0.113 | 0.024 |

No. 1-49 cells produce FR267471 specific mAbs.
No. 54, 55, 57, 65, 72, 81, 83, 108, 118, 122, and 132 hybridoma cells produce mAbs against both FR267471 and FR266831.
The selected 8 hybridoma cells (No. 7, 9, 14, 18, 20, 24, 28 and 31) which was competitive with FK778 are underlined.

5. First Limiting Dilution

After the first limiting dilution, 3 clones (designated No. A, B and C) were selected (Table 6).

TABLE 6
OD value at 490 nm of limiting dilution for selected clones
from competition test for each hybridoma using FK778

| No. | Clone No. | Pos | Neg |
|---|---|---|---|
| 7 | 7A | RO | −0.032 |
| | 7B | RO | −0.190 |
| | 7C | RO | −0.064 |
| 9 | 9A | RO | −0.163 |
| | 9B | RO | −0.161 |
| | 9C | RO | −0.163 |
| 14 | 14A | 2.713 | −0.075 |
| | 14B | 2.385 | −0.072 |
| | 14C | 2.026 | −0.082 |
| 18 | 18A | RO | 0.467 |
| | 18B | RO | 0.466 |
| | 18C | RO | 0.168 |
| 20 | 20A | 0.603 | −0.029 |
| | 20B | 0.883 | −0.027 |
| | 20C | 2.054 | −0.024 |
| 24 | 24A | RO | 0.077 |
| | 24B | RO | 0.003 |
| | 24C | RO | 0.009 |
| 28 | 28A | RO | 0.035 |
| | 28B | RO | 0.051 |
| | 28C | RO | 0.070 |
| 31 | 31A | RO | 0.033 |
| | 31B | RO | 0.021 |
| | 31C | RO | 0.028 |

Pos: FK778 (FR267471)-BSA plate,
Neg: FK778 (FR266831)-BSA plate
RO: out of range.

6. Secondary Limiting Dilution

No. A clone was subcloned. The result was 3 subclones (designated No. 1, 2 and 3). These clones were each cultured in 4 wells of a 24-well plate for clone, frozen and stored (Table 7). Cell Stock Media (IBL No. 34001) including 10% DMSO and 30% FBS in IBL Media I (IBL No. 33201) was used.

7. Cross-reactivity of the Active Metabolite FR271764

Eight No. A1 clones were tested for reactivity to FR267471-BSA and competition with exogenously added FK778 or FR271764 (Table 8). EC50 values were estimated from concentration-response binding curves by numerically fitting to an inhibitory effect sigmoidal Emax model defined in Eqs 1 using the nonlinear regression analysis program WINNONLIN (Pharsight Co., Ltd.).

$$Binding ( B / B 0 ) E max \cdot ( 1 - ( \frac{FR {concentartion}^{ γ}}{FR {concentration}^{ γ} + EC {50}^{γ}} ) ) \qquad \text{-} Eqs 1$$

The cross-reactivity of FR271764 of each clone was estimated from comparison of EC50 values between FK778 and FR271764 (Table 9) given in the following Eqs 2.

$$Cross \text{-} reactivity ( \% ) = \frac{EC 50 of FR 271764}{EC 50 of FR 238778} \times 100 \qquad \text{-} Eqs 2$$

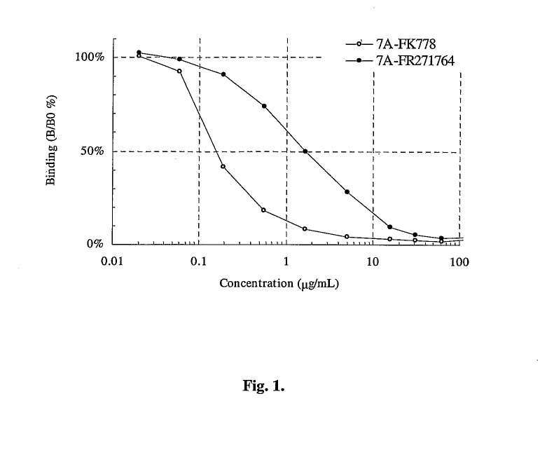

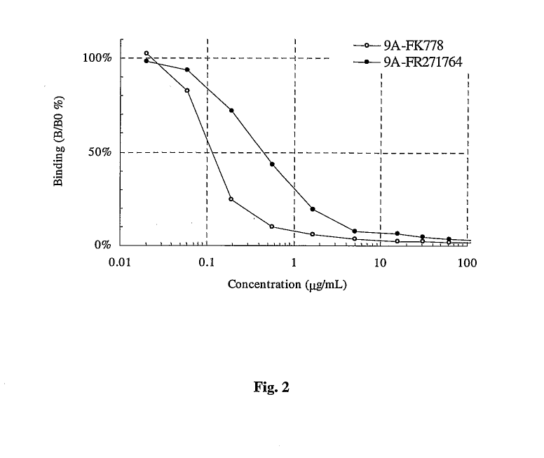

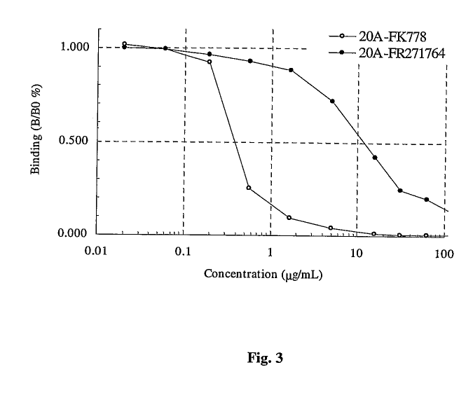

The cross-reactivity of FR271764 for the 3 clones 7A1, 20A1 and 9A1 was 10%, 3% and 27%, respectively (FIG. 1-3).

TABLE 8
B/B0 value of diluted subclones which were tested for reactivity
to FR267471-BSA and competition with exogenously added FK778
or FR271746 (active metabolite of FK778, M3)

| Subclone | | | | | | | | | | |
|---|---|---|---|---|---|---|---|---|---|---|
| No. | 125 | 62.5 | 31.25 | 15.625 | 5 | 1.67 | 0.56 | 0.19 | 0.06 | 0.02 |

Concentration of FK778 (μg/mL)

| 7A1 | 0.029 | 0.018 | 0.021 | 0.029 | 0.039 | 0.082 | 0.179 | 0.413 | 0.925 | 1.004 |
|---|---|---|---|---|---|---|---|---|---|---|
| 9A1 | 0.017 | 0.019 | 0.022 | 0.025 | 0.034 | 0.061 | 0.102 | 0.245 | 0.827 | 1.024 |
| 14A1 | 0.004 | 0.002 | 0.000 | 0.001 | 0.003 | 0.004 | 0.010 | 0.019 | 0.672 | 0.930 |
| 18A1 | 0.128 | 0.124 | 0.132 | 0.143 | 0.084 | 0.116 | 0.020 | 0.450 | 1.004 | 1.025 |
| 20A1 | 0.005 | 0.007 | 0.007 | 0.012 | 0.038 | 0.096 | 0.250 | 0.924 | 0.994 | 1.017 |
| 24A1 | 0.001 | 0.000 | 0.000 | 0.000 | 0.003 | 0.008 | 0.016 | 0.055 | 0.858 | 0.988 |
| 28A1 | 0.002 | 0.001 | 0.002 | 0.003 | 0.005 | 0.009 | 0.026 | 0.091 | 0.935 | 1.014 |
| 31A1 | 0.009 | 0.008 | 0.007 | 0.008 | 0.034 | 0.084 | 0.195 | 0.454 | 0.983 | 1.025 |
| | Concentration of FR271764 (μg/mL) | | | | | | | | | |
| 7A1 | 0.042 | 0.037 | 0.056 | 0.093 | 0.279 | 0.500 | 0.631 | 0.905 | 0.991 | 1.021 |
| 9A1 | 0.027 | 0.033 | 0.046 | 0.059 | 0.076 | 0.196 | 0.430 | 0.721 | 0.934 | 0.984 |
| 14A1 | 0.002 | 0.002 | 0.003 | 0.007 | 0.011 | 0.040 | 0.122 | 0.334 | 0.749 | 0.889 |
| 18A1 | 0.246 | 0.295 | 0.358 | 0.423 | 0.444 | 0.657 | 0.847 | 0.932 | 1.009 | 1.018 |
| 20A1 | 0.121 | 0.198 | 0.248 | 0.422 | 0.721 | 0.881 | 0.931 | 0.967 | 0.992 | 1.001 |
| 24A1 | 0.002 | 0.003 | 0.004 | 0.010 | 0.037 | 0.096 | 0.249 | 0.502 | 0.901 | 0.979 |
| 28A1 | 0.005 | 0.005 | 0.007 | 0.012 | 0.043 | 0.114 | 0.299 | 0.578 | 0.941 | 0.989 |
| 31A1 | 0.021 | 0.035 | 0.045 | 0.080 | 0.213 | 0.449 | 0.721 | 0.920 | 0.996 | 1.076 |

TABLE 9
Cross-reactivity of FR271764 (active metabolite of FK778, M3)

| Subclone | EC50 of FK238778 | EC50 of FR271764 | Cross-reactivity |
|---|---|---|---|
| No. | (μg/mL) | (μg/mL) | (%) |
| 7A1 | 0.154 | 1.533 | 10% |
| 9A1 | 0.108 | 0.405 | 27% |
| 14A1 | 0.075 | 0.134 | 56% |
| 18A1 | 0.169 | 3.014 | 6% |
| 20A1 | 0.394 | 12.143 | 3% |
| 24A1 | 0.095 | 0.191 | 50% |
| 28A1 | 0.109 | 0.241 | 45% |
| 31A1 | 0.171 | 1.150 | 15% |

Purification of mAbs from Mouse Ascites Fluid
1. Harvest of Ascites Fluid

30 BALB/c mice were injected with 0.2 mL/mouse of pristane (2,6,10,14-Tetramethylpentadecane, T7640, Sigma) into intraperitoneal, followed by bleeding for 3 weeks. Hybridomas (clones 7A1, 20A1, 9A1) were cultured in TIL medium (No. 33612, IBL) supplemented with 10% Fetal Calf Serum, harvested and injected into the intraperitoneal cavity of mice injected with pristane (2×10⁷ cells/mL×0.5 mL/mice). After injection of hybridomas, mice were bled for 10-12 days. After swelling of the abdomen, ascites fluid was obtained and centrifuged at 3000 rpm for 5 min, then stored at −20° C. The volume of ascites fluid was 50 mL for 7A1 and 20A1, and 25 mL for 9A1.

2. Purification of mAbs from Ascites Fluid

mAbs were purified by affinity chromatography with a HiTrap proteinA-HP column (Amersham Pharmacia Biotech, Uppsala, Sweden) Ascites fluid was diluted with 2 volumes of binding buffer (1.5M glycine buffer (pH8.9) containing 3M NaCl) and applied onto the HiTrap protein A-HP column equilibrated with 10 column volumes of binding buffer. After washing with binding buffer, antibodies were eluted with elution buffer (0.1M succinic buffer (pH4.0)). The antibody-containing fraction was dialyzed against 100 volumes of Dalbecco's phosphate buffered saline without Ca²⁺ and Mg²⁺ (D-PBS, No. 33273, IBL) with 2 replacements, using Seamless Cellulose Tubing (MWCO; 14,000, Sanko Junyaku, Japan) and stored at −20° C.

3. Estimation of Concentration and Purity of mAbs

Concentrations of each mAb was determined by absorbance at 280 nm using ε=1.38 (0.1%, 1 cm) and a Mr=150000 for IgG.

The value of absorbance at 280 nm divided by 1.38 produces the value for the concentration of antibody in mg/mL. The concentration of 7A1, 9A1 and 20A1 was 2.23 mg/mL, 2.58 mg/mL and 2.63 mg/mL, respectively.

Purity of each mAb was determined using gel-filtration chromatography with a Superdex 200 column (Amersham Pharmacia Biotech, Uppsala, Sweden). A 100 μL aliquot of mAb solution was applied to the Superdex 200 column equilibrated with D-PBS, run at a flow rate of 0.75 mL/min for 50 min and monitored by absorbance at 280 nm. The percentage of peak area of the IgG fraction was calculated using UNICORN software (Amersham Pharmacia Biotech, Uppsala, Sweden) and purity estimated for each mAb. The purity of 7A1, 9A1 and 20A1 was 96.84%, 97.50% and 80.32%, respectively.

Isotypes of 7A, 9A and 20A Clones

Isotypes of these mAbs were identified as IgG (1) heavy chain and kappa light chain.

Results

Hyperimmune Balb/c mice were used for the production of mAbs. Mice were immunized 7 times, followed by a final boost, and then spleen cells were collected and fused with X63-Ag8-653 myeloma cells in the presence of PEG. Hybridoma cell lines secreting antibody capable of binding to FK778 substance with high titer were selected by enzyme-linked immunosorbent assay (ELISA), and then subcloned using limiting dilution. 3 murine hybridoma producing anti-FK778 mAb were obtained and these clones were designated as 7A1, 9A1 and 20A1. The hybridoma 7A1, 9A1 and 20A1 (Identification: Mouse-Mouse hybridoma FK778-7A1, Mouse-Mouse hybridoma FK778-9A1 and Mouse-Mouse hybridoma FK778-20A1, respectively) were deposited as FERM ABP-10260, FERM ABP-10261 and FERM ABP-10262 respectively at the Patent Organism Depository Center, the National Institute of Advanced Industrial Science and Technology (AIST Tsukuba Central 6, 1-1-1, Higashi, Tsukuba-shi, Ibaraki 305-8566, Japan) on the date of Feb. 23, 2005. Isotypes of these mAbs were identified as IgG-(1) heavy chain and kappa light chain. mAbs of high purity were obtained by affinity chromatography. Purity analysis of the mAbs was performed by gel-filtration chromatography. Concentration of the mAbs was determined by absorbance at 280 nm using ε=1.38 (0.1%, 1 cm) and a Mr=150000 for IgG. The purity of 7A1, 9A1 and 20A1 was 96.84%, 97.50% and 80.32%, and concentration 2.23 mg/mL, 2.58 mg/mL and 2.63 mg/mL, respectively. The cross-reactivity of FR271764 for the 3 clones 7A1, 20A1 and 9A1 was 10%, 3% and 27%, respectively.

1. An antibody capable of binding to the FK778 substance.

2. The antibody of claim 1, which is a polyclonal antibody.

3. The antibody of claim 2, wherein the class of said polyclonal antibody is IgG.

4. The antibody of claim 1, which is a monoclonal antibody.

5. The antibody of claim 4, which is a monoclonal antibody produced by a hybridoma cell line resulting from cell fusion between an cell producing antibodies capable of the FK778 substance from an animal and a myeloma cell.

6. The antibody of claim 5, wherein said cell producing antibodies capable of the FK778 substance is a spleen cell line.

7. The antibody of claim 5 wherein said animal is a mouse.

8. The antibody of claim 4, wherein said class of the monoclonal antibody is IgG.

9. A highly-sensitive immunoassay method for the FK778 substance, which comprises immobilizing an antibody capable of binding to the FK778 substance, allowing the FK778 substance contained in a sample and the FK778 substance labeled by detectable substance to react competitively with said immobilized antibody and detecting said labeled substance bound to said immobilized antibody.

10. A highly-sensitive immunoassay method for the FK778 substance, which comprises using a first antibody capable of binding to the FK778 substance and an immobilized second antibody capable of binding to said first antibody, allowing the FK778 substance contained in a sample and the FK778 substance labeled by a detectable substance to react competitively with said first antibody and detecting said labeled FK778 substance bound to said first antibody bound in turn to said second antibody.

11. A highly-sensitive immunoassay method for the FK778 substance, which comprises using a first antibody labeled by a first detectable substance, which is capable of binding to the FK778 substance, the FK778 substance labeled by a second detectable substance, and an immobilized second antibody capable of binding to said second detectable substance, allowing said FK778 substance contained in a sample and the FK778 substance labeled by said second detectable substance to react competitively with said first antibody and detecting said first antibody bound to said labeled FK778 substance whose said second detecting substance is bound to said second antibody.

12. The highly-sensitive immunoassay method of claim 9, wherein said first antibody is a polyclonal antibody.

13. The highly-sensitive immunoassay method of claim 9, wherein said first antibody is a monoclonal antibody.

14. The highly-sensitive immunoassay method of claim 9, wherein said second antibody is immobilized on a plate.

15. A test kit for assaying the amount of the FK778 in a sample, comprising an antibody capable of binding to the FK778 substance and the FK778 substance labeled by detectable substance.

16. The test kit of claim 15, wherein said antibody capable of binding to the FK778 substance is a monoclonal antibody.

17. The test kit of claim 15 which further comprises a known quantity of the FK778 substance as a standard.

18. The test kit of claim 15, which further comprises an antibody, which can bind to said antibody capable of binding to the FK778 substance, or an antibody, which can bind to said detectable substance labeling said FK778 substance.
